(19) United States
(12) Patent Application Publication
(10) Pub. No.: US 2004/0243101 A1
(43) Pub. Date: Dec. 2, 2004

(54) Minimally invasive drug delivery catheter

(76) Inventor: Edward M. Gillis, San Jose, CA (US)

Correspondence Address: DURECT CORPORATION, 10240 BUBB ROAD, CUPERTINO, CA, 95014, US

(21) Appl. No.: 10/482,267

(22) PCT Filed: Jul. 2, 2002
(86) PCT No.: PCT/US02/21045

(51) Int. Cl. A61M 25/00

(52) U.S. Cl. 604/523

(57) ABSTRACT

The catheter of the invention is of a design adapted to be implanted, and fixed in place, without the use of a trocar or cannula, such that tissue damage is minimized both at the time of implantation, and later, over the period that the catheter remains implanted. In one embodiment, the catheter may be used to deliver a drug over a prolonged period of time, to a precise location, for instance to treat an inoperative tumor of the brain stem. In another embodiment, the catheter of the invention may be implanted, and maintained in place and used to deliver repeated doses of a drug to the same target over time without the necessity of repeatedly re-implanting a needle through tissue to access the target site.

### RELATIONSHIP TO OTHER APPLICATIONS

This application claims priority to U.S. provisional application 60/301,631 filed Jun. 27, 2001.

### FIELD OF THE INVENTION

The invention relates to minimally invasive catheters and methods for long-term delivery of a drug to a highly sensitive and delicate area of the body, such as the brain, particularly to the brain stem. More particularly, the invention relates to a catheter that may be used for long-term delivery of chemotherapeutic agents to the brain stem of a human patient.

### BACKGROUND OF THE INVENTION

The use of catheters and needles to deliver drugs to various parts of the body is well known, as is the direct injection of chemotherapeutic agents to treat disease. The obvious advantage of direct delivery of a drug over systemic delivery is that one avoids generalized toxicity. Delivery to a specific organ requires less drug and avoids non-specific toxicity and toxicity to inappropriate organs, such as hepatotoxicity and nephrotoxicity. Cancer chemotherapeutic agents, for example carboplatin and tamoxifen, are well known to cause general toxicity, nausea, vomiting, and reduction in bone marrow function that can result in anemia, risk of bruising or bleeding, and infection. Such undesirable side effects may be considerably reduced or eliminated if a lower dose is used and delivered directly to the tumor site.

Direct delivery of drugs, however, has certain difficulties and disadvantages, not least of all, those associated with local tissue destruction. Because catheters and needles need to be inserted through tissue to their target site, they inevitably cause some local tissue destruction. A non-rigid catheter is customarily inserted through tissue using a rigid cannula. The cannula is simply a hollow tube that is used to penetrate tissue, through which a catheter may be threaded. The cannula, of course, must have a diameter that is greater than that of the catheter. The volume of tissue destroyed is generally proportional to the diameter of the needle or cannula. Movement of the needle or cannula, once inserted, will cause additional destruction.

Additionally, wafers may be implanted at the site of the debulked tumor. Such wafers are generally hard discs made of poly-lactate, that slowly dissolve and release a chemotherapeutic agent that destroys cells locally at the tumor site (e.g., the GLIADEL® wafer used to treat glioblastoma multiforme). A disadvantage of such tumors however, is that they cause an inflammatory response that is, obviously, undesirable. Also, such water treatments only appear to increase life-span by a few months, at most.

Many organs such as the skin or liver are relatively insensitive to this degree of destruction, and the functioning of the organ is not disturbed by the insertion of a needle or cannula. Other tissue however, such as brain tissue, the tissues of the eye or any tissue very rich in nerve cells, such as spinal tissue or nerve plexus tissue, may be very sensitive to even a small amount of tissue destruction. The insertion of a needle or cannula into the brain tissue, such as the brain stem, may cause a fatal disruption in function. Thus it is well known in the art that direct delivery of drugs to these sensitive tissues is a very difficult and risky procedure. In pediatric oncology, the problems of chemotherapeutic drug delivery to the brain stem to treat inoperable tumors are well known. Treatment is generally achieved using systemic chemotherapy, with all its horrific side effects, and still the prognosis for such patients is very poor. (See Walter A W, et al., “Tamoxifen and carboplatin for children with low-grade gliomas: a pilot study at St. Jude Children's Research Hospital.” J Pediatr Hematol Oncol. 2000 May-June; 22(3): 247-51; and Puchner M J, et al., “Surgery, tamoxifen, carboplatin, and radiotherapy in the treatment of newly diagnosed glioblastoma patients.” J Neurooncol. 2001 September; 49 (2): 147-55).

Continual, long-term delivery of drugs to the brain faces the same hurdles as bolus delivery, plus the additional problems of implanting a delivery device into neural tissue and keeping it there, accurately and securely implanted, without causing further damage, and while delivering an appropriate dose of drug to the target site. Other difficulties include the risk of infection, especially when a catheter or needle is threaded from outside to inside the body, and the discomfort and inconvenience of an apparatus that may include a bulky external reservoir or pump.

Because of the difficulties of sustained delivery of drugs to brain tissue, the current treatments generally employ delivery of a bolus of drug, often with repeated doses. This method has two inherent disadvantages. First, repeated delivery requires repeated access with a concomitant likelihood of increased tissue damage. Secondly, and very importantly, the amount of drug delivered as a bolus is often much higher than the average amount of drug that a clinician would ideally like to deliver (a “super-optimal dose”). Thus the initial concentration of the drug, and therefore the (undesirable) toxicity of the drug bolus, is higher than would be ideally desirable, since the concentration of drug at the target site will be at its maximum initially, and will decrease over time. In contrast, continuous delivery of a drug would allow a lower dose to be delivered, at a steady rate, over a period of time, thereby reducing toxicity due to high initial drug concentrations. Continuous delivery would also reduce the need for repeated access to the target site, therefore reducing local tissue destruction.

There have also been some limited animal studies that involve the implantation of sustained drug delivery microspheres into brain tissue to treat glioma. (Emerich D F et al., “Injectable chemotherapeutic microspheres and glioma I: enhanced survival following implantation into the cavity wall of debulked tumors.” Pharm Res 2000 July;17(7):767-75). In this case, injectable polymeric microspheres, formulated to release carboplatin or BCNU for 2-3 weeks were implanted into the cortex of rats. These experiments appear to indicate that that sustained delivery of chemotherapy is superior to equipotent bolus doses following tumor resection.

Current methods used to deliver drugs to the brain of human patients typically use a needle that can penetrate to the area most desired for drug delivery. The needle used in the current methods has a hub fixed to the distal end that limite the depth to which it can be inserted. This configuration does not allow for the routing of the rigid needle hub to a remote area for continuous delivery via an implanted device. In addition, a fully rigid configuration would be subject to movement from the area of implantation due to forces through bending and flexing that could be applied to the device from typical movement of the subject.

Another currently employed method employs a cannula that is used to penetrate the tissue and access the desired area of interest. This device then allows for the introduction of a flexible catheter that contains a stylet through the center lumen of the cannula. The cannula can then be removed and the flexible catheter routed to the desired area of implantation. This method suffers the disadvantage of having to use a cannula to place the catheter, which causes additional tissue damage.

In summary, there is a need for a catheter that can be used to deliver a drug to a precise location, in a delicate and sensitive area of the body, such as the brain stem, in a minimally invasive fashion. There is a need for such a catheter that can be implanted without the use of a trocar or cannula, such that tissue damage is minimized both at the time of implantation, and later, over the period that the catheter remains implanted. There is a need for an implanted catheter that may be used for repeated bolus delivery of a substance, or for continuous delivery. There is a need for such a catheter that is adapted for delivery of a drug, continuously over a period of time, for example to treat an inoperative tumor of the brain stem. There is a need for such a device that can be implanted, and thereby eliminate the need for repeated access to the target site as currently required by bolus injection, and that would also reduce the need to deliver super-optimal initial doses of a drug, as currently required by bolus injection. The present methods and devices do not adequately address these needs, which is a long-felt need, particularly in pediatric oncology. At present, the prognosis for children with inoperative cancers of the brain stem is very bleak. The present invention addresses this pressing need, and has been shown to be effective in vitro using animal studies.

### SUMMARY OF THE INVENTION

The invention encompasses a catheter with a flexible portion that may be attached to a pump or drug reservoir, and a rigid tip portion that is used to penetrate a delicate and sensitive tissue, such as the brain stem, in a minimally invasive fashion and thereby deliver a drug either as a bolus, or as repeated bolus delivery, or continuously over a period of time. The catheter is adapted to be implanted, and fixed in place, without the use of a trocar or cannula, such that tissue damage is minimized both at the time of implantation, and later, over the period that the catheter remains implanted. The catheter may be placed by drilling a hole through the posterior aspect of the cranium to gain access to the brain stem. Only a very small hole need be made, which is advantageous over a larger hole tat would be required to debulk a tumor. The catheter is adapted to deliver a drug to a precise location, for instance to treat an inoperative tumor of the brain stem. The present invention provides a device that can be implanted. A substance such as a drug may be delivered either as a bolus, or as repeated/preiodic boluses, or by continual delivery. Repeated bolus delivery may be facilitated by use of an access port attached to the proximal end of the catheter, that may be repeatedly accessed by a syringe and needle. The present invention thereby eliminates the need for repeated access to the target site as currently required by bolus injection, and that also reduces the need to deliver super-optimal initial doses of a drug, as currently required by bolus injection.

A formal description of one of the embodiments of the invention may be summed up as follows: A catheter designed for the continuous delivery of a fluid into a target site, said catheter comprising a tube having a flexible portion, said flexible portion having a proximal end and a distal end, and a rigid tip, said rigid tip having a proximal end and a distal end, and wherein said tip is either open at its distal end or is fenestrated, and wherein said proximal end of said flexible portion is communicably attached to a drug delivery apparatus, and wherein said distal portion of said flexible portion is communicably attached to said rigid tip, and wherein said tip is adapted for implantation into a tissue, and wherein said tip is further adapted to minimize damage to said tissue into which it is implanted and wherein a fluid may be delivered from said drug delivery apparatus via said flexible portion and thence to said tip, whereby said fluid is released from said tip into said target site.

### DETAILED DESCRIPTION OF THE DRAWINGS

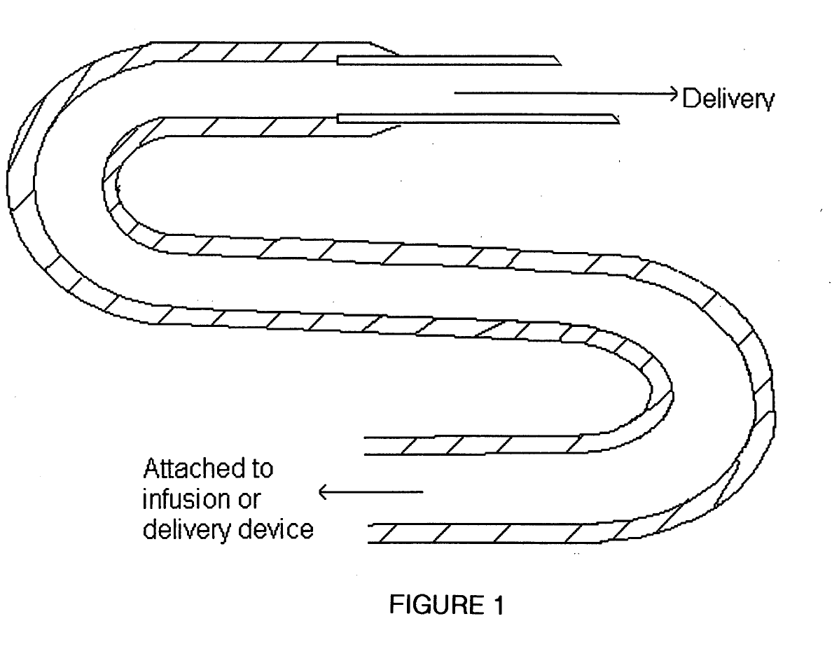

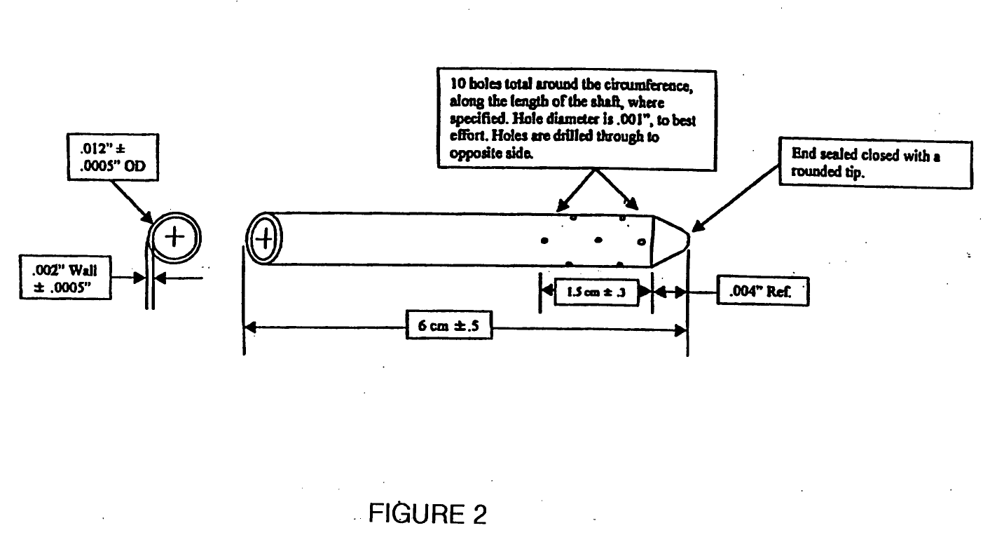

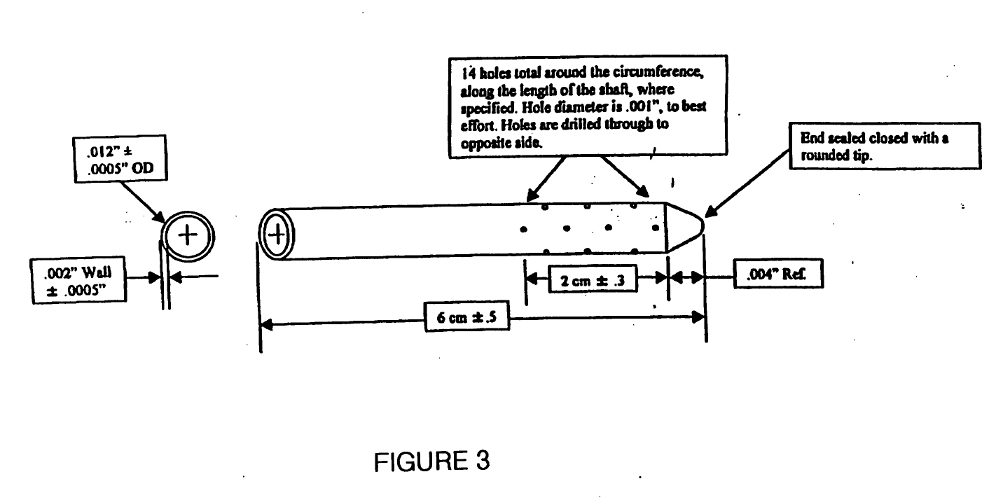

FIG. 1. A general embodiment (not to scale) of the minimally invasive catheter of the invention. Although a sharpened open tip is shown, the tip may equally have a closed distal end but be fenestrated as shown in FIG. 2 and FIG. 3.

FIG. 2. Engineering drawing of a fenestrated catheter tip with 1.5 cm spread.

FIG. 3. Engineering drawings of a fenestrated catheter tip with 2 cm spread.

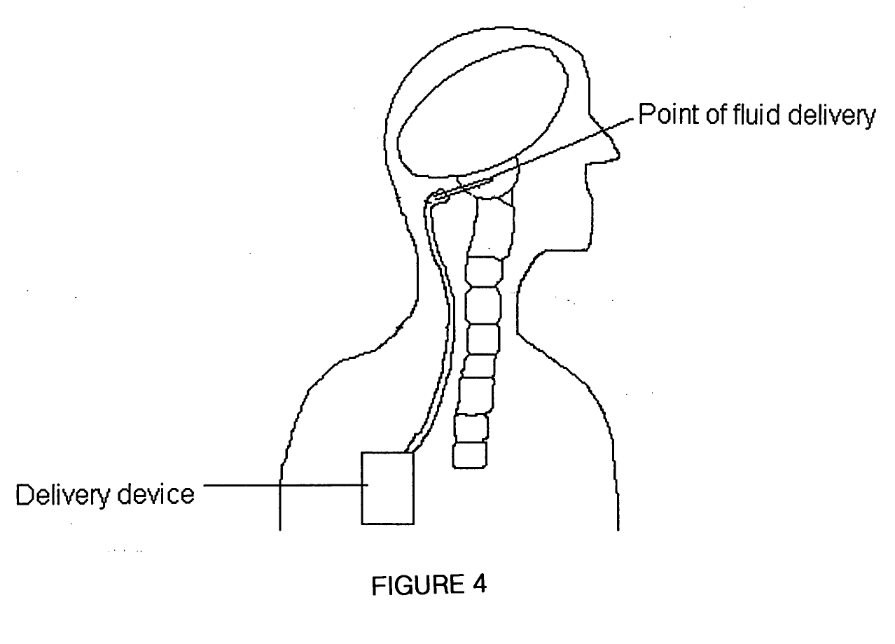

FIG. 4. Schematic diagram of the catheter placed in the brain stem of a human.

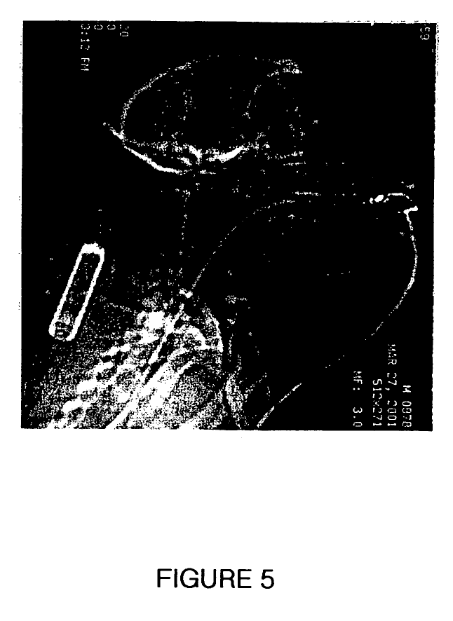

FIG. 5. CAT-scan showing caterer implanted into the brain stem of a cynomologous monkey. The catheter is attached to an osmotic pump clearly visible, implanted subcutaneously between the shoulder blades. In this experiment, saline was delivered continuously for a period of three months at a rate of 0.41 microliters per hour (about 10 microliters per day). No ill effects were observed in the subjects.

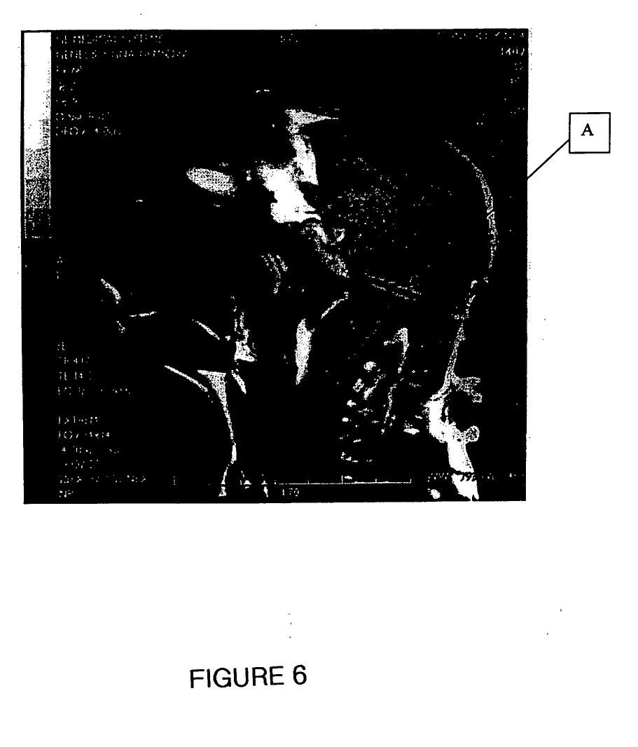

FIG. 6. CAT-scan showing clearly showing caterer tip (A) implanted into the brain stem of a cynomologous monkey.

### DETAILED DESCRIPTION OF THE INVENTION

Definitions

“Drug” may include any substance meant to alter body physiology, such as to treat a disease, such as cancer. For example, a drug may be selected from the following types of substances: an anti-cancer chemotherapeutic agent (such as carboplatin and tamoxifen), an antibody (such as an antibody that binds to a cancer-associated growth hormone receptor), a peptide, protein, carbohydrate, nucleoprotein, mucoprotein, lipoprotein, synthetic polypeptide or protein, or a small molecule linked to a protein, glycoprotein, steroid, nucleic acid (e.g., DNA, including cDNA, or RNA, or a fragment thereof), nucleotide, nucleoside, oligonucleotides (including antisense oligonucleotides), gene, lipid, hormone, vitamin, or combination thereof. A drug may include immunosuppressants, antioxidants, anesthetics, chemotherapeutic agents, steroids (including retinoids), hormones, antibiotics, antivirals, antifungals, antiproliferatives, anticoagulants, antiphotoaging agents, melanotropic peptides, nonsteroidal and steroidal anti-inflammatory compounds, antipsychotics, and radiation absorbers (such as molecules designed for use in photo-dynamic therapy to treat cancers), including UV-absorbers. A drug may also include anti-infectives such as nitrofurazone, antibiotics, including penicillin, tetracycline etc; anti-virals including idoxuridine; antiallergenics such as antazoline, hydrocortisone etc; miotics and anticholinesterases such as pilocarpine, esperine salicylate etc; sedatives and hypnotics such as pentobarbital sodium; progestational agents such as progesterone, or megestrol; humoral agents such as the prostaglandins, for example PGE₁ and PGF₂; antispasmodics such as atropine; natural and synthetic bioactive peptides and proteins, including growth factors, cell adhesion factors, cytokines, and biological response modifiers. A drug may also include a vaccine and the substance to be delivered is an antigen. A drug may also include a narcotic analgesic, such as sufentanil and fentanyl and congeners thereof.

“Continuous delivery” means delivery of a substance over a period of time such that the procedure is distinguished from “bolus” delivery. Continuous delivery generally involves delivery of a substance over a period of time without interruption. The rate of delivery need not be constant, and the period of delivery need not be very long, ie: the period of constant delivery may be over a period of maybe half an hour or an hour or a few hours, but may also be over a period of days, weeks, months, or even a year or more.

“Implanted” means placed within the body, and maintained at that location for some extended period of time. The period of time during which the implanted object is maintained in place will be generally considerably greater than that customarily required to introduce a bolus of a substance, such as a drug. Normally, injection of a drug takes only a few seconds to about a minute. Therefore a device that is placed in the body for any extended period greater about a few (say two) minutes could reasonably be considered to be implanted for purposes of this disclosure. For example, the catheter of the invention may be placed subcutaneously, or within a tissue or organ such that the catheter so implanted is intended to remain at the site of implantation for some time, at least for, say, half-an-hour, or an hour or more. In some embodiments the catheter may be implanted for days, weeks, months or even longer. Some of the drug delivery apparatuses that may be used with the catheter of the invention, for example the osmotic pumps, are designed to be implanted for periods greater than a month and to deliver drug during this period. A drug delivery apparatus may be implanted, for example, subcutaneously, or within a tissue or organ, or within a body cavity such as the peritoneal cavity, the thoracic cavity, the pelvic cavity, or within the bladder, uterus, or any other cavity or location that is convenient for delivery of the intended substance. A catheter may be implanted into a tissue, for example into brain tissue, and may be affixed in place by fixing the catheter to another tissue, such as bone or cartilage, using an adhesive or screws, clamps, sutures or any other suitable fixing means.

“Target site” means the site for intended delivery of a substance, for example the target site of an anti-tumor drug may be a tumor located within a tissue, or may be tissue in proximity to the tumor. In the case of delivery of a neurotropic substance to the brain, the target site may be the site of traumatic damage or a region of tissue where neurodegenerative pathology is present, such as in the treatment of Alzheimer's or Parkinson's disease.

“Proximal end” is a relative term, and generally refers to the end of a device, such as a catheter, than is nearest to the operator (i.e. the surgeon) and is furthest away from the treatment site. In the present invention the flexible portion of the catheter has a proximal end that may be communicably attached to an access port or drug delivery apparatus, such as a pump, or reservoir.

“Distal end” is a relative term and generally refers to the end of a device, such as a catheter, that is furthest away from the operator (i.e. the surgeon) and is closest to the treatment site. In the present invention the distal end of flexible portion of the catheter may be communicably attached to a rigid tip that is used to penetrate tissue.

“Nervous tissue” includes, but is not limited to brain tissue, the tissues of the eye or any tissue rich in nerve cells, such as spinal tissue or nerve plexus tissue.

“Drug delivery apparatus” includes but is not limited to a syringe, a drug reservoir or a pump of any kind, for example an osmotic pump, an electromechanical pump, an electroosmotic pump, an effervescent pump, a hydraulic pump, a piezoelectric pump, an elastomeric pump, a vapor pressure pump, or and an electrolytic pump. Such a pump may be externally worn or may be implanted within the body.

### DESCRIPTION

The catheter of the invention is of a design adapted to be implanted, and fixed in place, without the use of a trocar or cannula, such that tissue damage is minimized both at the time of implantation, and later, over the period that the catheter remains implanted. In one embodiment, the catheter may be used to deliver a drug over a prolonged period of time, to a precise location, for instance to treat an inoperative tumor of the brain stem. In another embodiment, the catheter of the invention may be implanted, and maintained in place and used to deliver repeated doses of a drug to the same target over time without the necessity of repeatedly re-implanting a needle through tissue to access the target site.

The present invention is particularly directed to delivery of drugs to the brain and nervous tissue. Delivery of drugs to such tissue is important to treat a number of diseases, such as cancers (Walter A W, et al., J Pediatr Hematol Oncol. 2000 May-June; 22(3):247-51), depression, epilepsy, psychosis, schizophrenia (see U.S. Pat. No. 5,975,085), pain, narcolepsy, tinnitus (see U.S. Pat. No. 6,676,655), neurodegenerative disorders such as Alzheimer's, Parkinson's etc (see U.S. Pat. No. 5,720,720), traumatic brain injury, and obesity (see U.S. Pat. No. 6,129,685). The present invention may also be used to deliver substances used for imaging in various organs, such as the delivery or radio-opaque substances or imaging substances used in MRI or other methods.

Drugs delivered to treat cancers would include, but are not limited to tamoxifen and carboplatin. Drugs delivered to treat epilepsy would include, but are not limited to phenytonin. Drugs delivered to treat psychosis would include, but are not limited to tri-cyclic anti-depressants such as chloromazine. Drugs delivered to treat schizophrenia would include, but are not limited to serotonin selective reuptake inhibitors (SSRI's) such as prozac. Drugs delivered to treat depression would include, but are not limited to SSRI's and 5-hydroxy-L-tryptophan. Drugs delivered to treat pain would include, but are not limited to fentanyl, sufentanil, morphine, and derivatives and congeners of such drugs. Drugs delivered to treat narcolepsy would include, but are not limited to dextroamphetamine sulfate. Drugs delivered to treat tinnitus would include, but are not limited to sodium channel receptor antagonists such as lidocane, GABA-A receptor agonists such as benzodiazopine, and MDNA receptor antagonists such as dextromethorphan. Drugs delivered to treat brain injury would include, but are not limited to steroids, and drugs that would stimulate nerve growth such as Brain Derived Neurotrophic Factor (BDNF), and MDNA receptor antagonists. Drugs delivered to treat neurodegenerative disorders may include, but are not limited to acetylcholine esterase inhibitors, such as Aricept, to treat Alzheimer's, and L-DOPA to treat Parkinson's disease. Additionally the current invention could be used to deliver agents used in gene therapy, such as, for example, naked polynucleotides encoding proteins that stimulate neuron growth, such as BDNF. Such polynucleotides could also be delivered using microspheres, liposomes, synthetic viral capsids, or virus vectors such as adenovirus, adeno-associated virus, lentivirus, herpes virus etc. Such vectors are well know in the field of gene therapy and could be used to deliver genes encoding any protein of therapeutic value. Additionally such vectors can be used to deliver anti-sense polynucleotides to alter translation of mRMA's thereby altering the expression of specific proteins.

The invention encompasses a catheter with a flexible portion that at the proximal end may be attached to a pump or drug reservoir, and a rigid tip portion at the distal end that is sufficiently rigid to penetrate tissue such that the tip can access an area of the body (i.e. the brain stem) in a minimally invasive fashion via direct penetration.

The catheter tip at the distal section may be a few centimeters in length (e.g.: from about 0.4 cm to about 12 cm) such that it is adapted to facilitate penetration of the tip to the desired area of drug delivery. The Tip must be of a suitable length so as to allow for the piercing and placement of it yet not so long as to make the Catheter difficult to route to the desired area of pump implantation. The tip of the catheter must be long enough to allow for penetration to the desired location of drug delivery yet not be so long as to not allow for routing it to the desired area. Generally the tip length will correspond to the distance from the outside of the organ in which the target is located to the target location within the organ. For example, a catheter designed to deliver a chemotherapeutic agent to a central area of the brain stem of a human being may have a tip of about 0.25-3 cm or optionally about 1.5-2.5 cm in length (e.g.: about 2.1 cm in length, which is the embodiment used experimentally) depending on exactly where the target point (tumor) is. If it were necessary to deliver a drug to the putainment, then a desirable length for the catheter tip may be about 6-12 cm in length. The target point may be at the site of a tumor, identified and located by x-ray, ultrasound, MRI or NMR etc. Positioning is commonly done using 3-D reconstruction of the brain from CAT scan images. The catheter tip may be positioned directly via stereotaxis or other means to the desired location. Positioning may be done using 3-D reconstruction of the brain from CAT scan images

The outer diameter of the catheter tip may be from about 0.1 mm to about 2.0 mm, or optionally about 0.1 mm to about 1.0 mm. (The experimental catheter was 0.25 mm in outer diameter). The inside diameter of the catheter may be from about 0.05 mm to about 0.75 mm.

The tip may be sharpened to facilitate penetration of the tissue, and may have an open lumen tip such that fluid may pass directly from the open end of the tip, or may be closed at the end, but be “fenestrated” such that fluid may pass out from a plurality of holes distributed along the length of or towards the end of the tip. The tip may be made of any number of reasonably rigid materials. Such materials may include, for example, metals (e.g.: steel, titanium, an alloy such as a nickel-titanium alloy), hard plastics and polymers (e.g.: polycarbonates, acetates etc), carbon-fiber composites, glass, etc. It is desirable to use a material that may be easily formed to provide the fine structure required, and may be easily sterilized, to allow for safe, aseptic implantation.

The proximal section of the catheter contains a substantially flexible section that allows for it to be indwelling and routed to a different area of the body and communicably attached to a reservoir or drug delivery apparatus such as a pump. The flexible section provides the connection between the pumping device and the rigid distal catheter Tip. The flexible proximal section may be made of any suitable flexible material such as, for example silicone rubber or polyurethane or low-density polyethylene. As with the tip, it is desirable that the flexible material be easily sterilized. Also, the proximal section (as with the tip) should be made of a biocompatible material, i.e., a material that is non-toxic to a recipient and present no significant, deleterious or untoward effects on the recipient's body.

The catheter may be anchored in place at the junction of the rigid and floppy sections so as to allow for the tip to remain fixed at the site of desired delivery. The proximal end can then be routed to the area of the body for attachment to the delivery device.

One preferred embodiment is for the delivery of drugs to the brainstem. The rigid section is made from a Nickel-Titanium (“NiTi”) Alloy and can be minimally invasively placed and the junction fixed in place at the back of the brainstem via a cyanoacrylate adhesive or other means for affixing. The catheter is ideally fixed at the proximal end of the rigid section or at the junction between the rigid and floppy section. The rigid section needs to be approximately 1 to 8 cm (more likely 2-5 cm) in length to allow for access to the preferred site of implantation within the stem yet not so long as to cause difficulty in routing the catheter down through the base of the neck. The floppy section, which is made of silicone rubber, is then routed through the neck to the middle of the back (between the shoulder blades) where the drug delivery apparatus is implanted. The floppy section needs to be of sufficient length so as to allow for routing to the desired location. This is typically 10-50 cm for a between the shoulder blades location. (See attached Figure) The length can be longer if the dispensing device needs to be located in another region of the body (such as the abdomen).

In another embodiment, the invention may be used to deliver drugs to the putainment of the brain. This embodiment would, or course, require a longer catheter tip, since the putainment is deep within the brain. In other embodiments, the invention may be used to deliver drugs to the spinal cord, or the eye or to any delicate area such as a nerve plexus.

The catheter of the invention may be adapted for use in long-term delivery of a drug, and for this purpose may be communicably attached to a drug reservoir and/or a pump. Many kinds of pump and/or drug reservoir could be used, including, for example an osmotic pump, an electromechanical pump, an electroosmotic pump, an effervescent pump, a hydraulic pump, a piezoelectric pump, an elastomeric pump, a vapor pressure pump, or and an electrolytic pump. Such a pump may be externally worn or may be implanted subcutaneously, or within tissues, at any convenient location in the body, for example between the shoulder-blades, such that the pump may effectively deliver a desired amount of a drug at a desired rate, via the catheter, to the target tissue. The rate of drug delivery may be varied as clinically appropriate. Practically any rate of delivery is possible depending on the pump used and may, for example be from about 0.01 microliters per day to about 2 milliliters per day. For a chemotherapeutic agent such as carboplatin or tamoxifen, a possible rate of daily delivery may be, for example 10 microliters per day. (See Walter A W, et al., “Tamoxifen and carboplatin for children with low-grade gliomas: a pilot study at St. Jude Children's Research Hospital.” J Pediatr Hematol Oncol. 2000 May-June; 22 (3): 247-51).

In certain embodiments, it may be advantageous to use an osmotic pump such as the Duros® pump, an osmotic pump designed for sustained delivery of a drug which provides advantages over the other pumps in that it is very small, easily implantable, and provides very accurate sustained release kinetics delivering very small amounts of drug over a period of weeks or months. Such accurate delivery of small drug doses is particularly important when using highly potent agents such as chemotherapeutic drugs.

In a particular embodiment, the rigid catheter tip is 2.5 cm in length. The floppy section is 25 cm long, is made of silicone rubber, and is routed through the neck to the middle of the back (between the shoulder blades) where the drug delivery apparatus is implanted. The catheter is attached to a drug delivery apparatus. The drug delivery apparatus is an osmotic pump filled with a pharmaceutically acceptable formulation of carboplatin. The catheter tip is implanted into the brain stem, and affixed in place by fixing it to the skull bone using a cyanoacrylate adhesive. The catheter tip is placed by stereotaxis, in close proximity to the target site, which is a tumor within the brain stem. The drug delivery apparatus and the catheter of the invention are implanted for a period of three months, during which time it delivers carboplatin at a rate of 10 microliters per day, thereby treating the tumor.

In Vivo Example

The catheter of the invention has been tested in vivo on primates. Two cynomologous monkeys were implanted with the catheter of the invention. The catheter used was a blunt-tipped catheter with side-pores extending 3-4 mm from the tip. The posterior aspect of the cranium was penetrated with a drill through the occipital bone, and then the catheter was placed through the cerebellum and fourth ventricle into the roof of the pons. The catheter tip was held in place by fixing it to the skull bone using cyanoacrylate adhesive. The floppy distal section of the catheter was routed subcutaneously through the neck to the middle of the back, and connected to an osmotic pump. See FIGS. 5 and 6. The osmotic pump was implanted subcutaneously between the shoulder blades. Saline was delivered from the pump, via the catheter continuously for a period of three months at a rate of 0.41 microliters per hour (about 10 microliters per day). No ill effects were observed in the subjects over the entire three-month period. This in vivo experiment shows that the catheter described herein can be successfully implanted into the brain stem of a mammal, and used to deliver a substance to the brain stem over a period of three months without any observable ill-effects.

1. A catheter designed for the continuous delivery of a fluid into a target site, said catheter comprising a tube having a flexible portion, said flexible portion having a proximal end and a distal end, and a rigid tip, said rigid tip having a proximal end and a distal end, and wherein said tip is either open at its distal end or is fenestrated, and wherein said proximal end of said flexible portion is communicably attached to a drug delivery apparatus, and wherein said distal portion of said flexible portion is communicably attached to said rigid tip, and wherein said tip is adapted for implantation into a tissue, and wherein said tip is further adapted to minimize damage to said tissue into which it is implanted and wherein a fluid may be delivered from said drug delivery apparatus via said flexible portion and thence to said tip, whereby said fluid is released from said tip into said target site.

2. The catheter of claim 1 wherein the rigid tip has a diameter of between about 0.05 mm to 1.0 mm.

3. The catheter of claim 2 wherein the rigid tip has a diameter of between about 0.1 mm to 0.75 mm.

4. The catheter of claim 2 wherein the rigid tip has a length of between about 0.4 cm to 12.0 cm.

5. The catheter of claim 4 wherein the rigid tip has a length of between about 0.5 cm to 3.0 cm.

6. The catheter of claim 2 wherein the rigid tip is adapted for insertion into the brain of a mammal.

7. A method for delivery of a chemotherapeutic agent to a tumor in the brain of a mammal, the method comprising placing the rigid tip of the catheter of claim 1 into the brain, at or near the tumor location, and delivering therefrom a chemotherapeutic agent.
